(19) United States
(12) Patent Application Publication
(10) Pub. No.: US 2014/0014116 A1
(43) Pub. Date: Jan. 16, 2014

(54) SYSTEMS AND METHODS FOR TREATMENT OF SLEEP APNEA

(71) Applicants: Edward M. Gillis, Livermore, CA (US); Andrew Poutiatine, Mill Valley, CA (US)

(72) Inventors: Edward M. Gillis, Livermore, CA (US); Andrew Poutiatine, Mill Valley, CA (US)

(21) Appl. No.: 13/939,107

(22) Filed: Jul. 10, 2013

Related U.S. Application Data
(60) Provisional application No. 61/671,643, filed on Jul. 13, 2012.

(51) Int. Cl. A61F 5/56

(52) U.S. Cl. CPC A61F 5/566
USPC 128/848

(57) ABSTRACT

A self-adjusting implant includes a first anchor configured to anchor to a first tissue location and apply a first tension force thereto; a second anchor configured to anchor to a second tissue location spaced apart from the first tissue location and apply a second tension to the second tissue location; a third anchor configured to anchor to a third tissue location spaced apart from and non-collinear with the first and the second tissue locations; a first tension-bearing segment spanning between the first and the third anchors, comprising a first deformable portion; and a second tension-bearing segment spanning between the second and the third anchors wherein the third anchor is configured to generally distribute a tension of the first tension-bearing segment with a tension of the second tension-bearing segment. The invention also includes methods for treating airway disorders that can be used with described implants.

### CROSS REFERENCE TO RELATED APPLICATIONS

This application claims priority to U.S. Provisional Application No. 61/671,643 filed on Jul. 13, 2012 which is herein incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD

The invention relates to the field of methods and devices for the treatment of obstructive sleep apnea, and more particularly to opening the airway of subjects with symptoms of obstructive sleep apnea.

### BACKGROUND

Sleep apnea is defined as the cessation of breathing for ten seconds or longer during sleep. During normal sleep, the throat muscles relax and the airway narrows. During the sleep of a subject with obstructive sleep apnea (OSA), the upper airway narrows significantly more than normal, and during an apneic event, undergoes a complete collapse that stops airflow. In response to a lack of airflow, the subject is awakened at least to a degree sufficient to reinitiate breathing. Apneic events and the associated arousals can occur up to hundreds of times per night, and become highly disruptive of sleep. Obstructive sleep apnea is commonly but not exclusively associated with a heavy body type, a consequence of which is a narrowed oropharyngeal airway.

Cyclic oxygen desaturation and fragmented sleeping patterns lead to daytime sleepiness, the hallmark symptom of the disorder. Further consequences of sleep apnea may include chronic headaches and depression, as well as diminished facilities such as vigilance, concentration, memory, executive function, and physical dexterity. Ultimately, sleep apnea is highly correlated with increased mortality and life threatening comorbidities. Cardiology complications include hypertension, congestive heart failure, coronary artery disease, cardiac arrhythmias, and atrial fibrillation. OSA is a highly prevalent disease conditions in the United States. An estimated 18 million Americans suffer from OSA to degrees that range from mild to severe, many of whom are undiagnosed, at least in part because the afflicted subjects are often unaware of their own condition.

Treatment of OSA usually begins with suggested lifestyle changes, including weight loss and attention to sleeping habits (such as sleep position and pillow position), or the use of oral appliances that can be worn at night, and help position the tongue away from the back of the airway. More aggressive physical interventions include the use of breathing assist systems that provide a positive pressure to the airway through a mask that the subject wears, and which is connected to a breathing machine. In some cases, pharmaceutical interventions can be helpful, but they generally are directed toward countering daytime sleepiness, and do not address the root cause. Some surgical interventions are available, such as nasal surgeries, tonsillectomy and/or adenoidectomy, reductions in the soft palate or the uvula or the tongue base, or advancing the tongue base by an attachment to the mandible and pulling the base forward. These surgical approaches can be quite invasive and thus have a last-resort aspect to them, and further, simply do not reliably alleviate or cure the condition. There is a need for less invasive procedures that show promise for greater therapeutic reliability.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1A-C show two embodiments of adjusting implant with two legs.

FIG. 2 shows an adjusting implant with a compliant elastic section attached to the fixed anchor.

FIG. 3 shows an adjusting implant with two self-adjustment points.

FIG. 4 shows an adjusting implant with a rocker leg instead of an eye bolt or pulley.

FIGS. 5A-E show an adjusting implant during normal food swallowing showing progression of the tongue wave.

FIG. 6A shows a normal tongue during sleep. FIG. 6B shows a tongue falling back when the body is in a sleep cycle. FIG. 6C shows a tongue, such as the one shown in FIG. 6B, during a sleep cycle, after implant of an adjusting implant.

FIG. 7 shows an adjusting implant with a looped adjusting element and an extended anchor section on the loop.

FIG. 8 shows an adjusting implant with hard stops to limit the amount of relative displacement between the two legs.

FIG. 9 shows an adjusting implant with a cam mechanism that creates a mechanical advantage between the two adjusting elements.

FIG. 10 shows an implant with 4 adjusting anchors and three cascading adjustable elements, utilizing x, y, and z-axes.

FIG. 11 shows a graph of several curves of force applied to the tongue as a function of displacement of the tongue.

FIG. 12A shows a mechanism that can be used to modulate a force. FIG. 12B shows a graph of force as a function of device displacement.

FIG. 13 shows another embodiment of an adjusting implant with a means to create a directionally controlled motion of the adjustable anchors.

FIGS. 14A-E show a one-piece implant during normal food swallowing and progression of the tongue wave, similar to that shown in FIGS. 5A-E

FIG. 15A shows a normal tongue during sleep. FIG. 15B shows a tongue falling back when the body is in a sleep cycle. FIG. 15C shows a tongue, such as the one shown in FIG. 15B, during a sleep cycle, after implant of an adjusting implant.

FIGS. 16A-B show two views of an implant with implant legs on same side of the centerline of the tongue and a fixed anchor on the centerline.

FIGS. 17A-B show two views of an implant with implant legs on opposite sides of centerline of the tongue and a fixed anchor off of the centerline.

FIGS. 18A-B show two views of an implant with implant legs on opposite sides of centerline and with a fixed anchor on the centerline of the tongue.

FIG. 19 shows the results of force exerted by implants when constrained and when rotated about a fixed point.

### DETAILED DESCRIPTION

The present invention includes methods and devices for implanting in or adjacent to an airway forming tissue to treat an airway tissue disorder, such as a breathing disorder. The methods and devices may be used for treating any sort of disorder, problem, or syndrome, including disordered breathing, hypopnea, sleep apnea, snoring, speech problems, and swallowing problems.

One aspect of the invention takes advantage of a characteristic of force that allows it to be redistributed, such as from a tissue to an implant or from one portion of an implant to another portion of an implant. An implant can be configured to provide sufficient force to have a therapeutic effect while reducing unwanted side effects.

In some embodiments, redistributing a force placed on an implant reduces a (peak) force experienced by the implant (or a portion of the implant) and/or reduces a (peak) force experienced by a body tissue. Reducing a force experienced by a body tissue may reduce or prevent tissue cutting, shearing, tearing or other tissue damage and/or may reduce or prevent tissue remodeling. Redistributing a force may reduce or prevent unwanted device movement or reduce or prevent device breakage, stress, strain, or other damage. Redistributing a force experienced by a tissue may reduce or relieve unwanted side effects including but not limited to discomfort or pain, difficulty swallowing, and/or difficultly speaking. An implant placed in a patient's body may be configured to self-adjust and thereby redistribute a force on it.

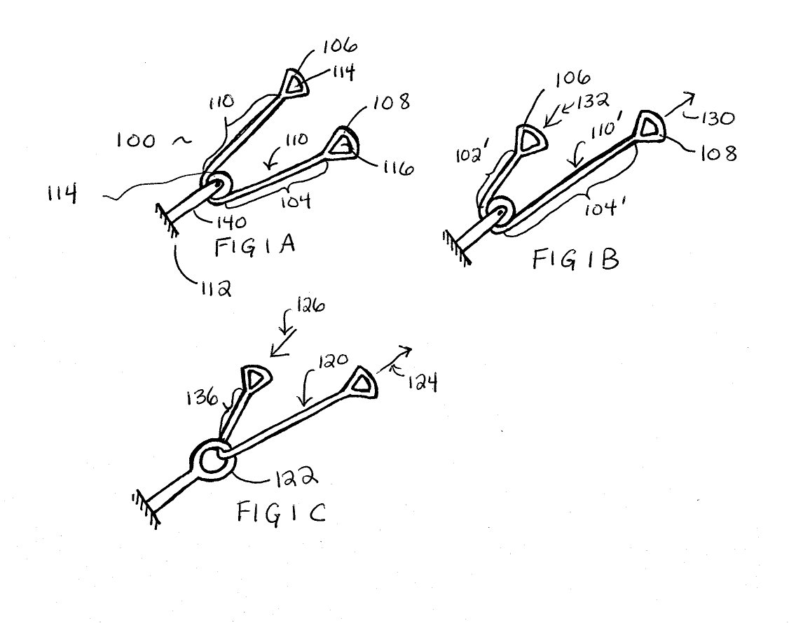

FIG. 1A-C shows examples of self-adjusting implants and methods of using self-adjusting implants to redistribute forces. FIGS. 1A-B shows implant 100 in two different configurations to apply force to a body tissue. Implant 100 shows element 110 with a first elongate leg 102 and a second elongate leg 104 that partially wraps around a pulley. Elongate member 110 could wrap partially around a pulley, or could wrap one or more times around a pulley mechanism. First anchor end 106 and second anchor end 108 have openings 114, 116 to allow tissue growth therethrough and to anchor the elongate legs into tissue, such as in two locations in a posterior tissue adjacent to an airway. The anchor ends may have one opening or may have multiple openings to allow tissue growth or may have no openings. In other embodiments, the anchor ends may be a solid material in any shape to hold the anchor in place. Any composition and any shape that allows the anchor to hold onto or be held by tissue can be used. For example, any of the anchor ends as described in U.S. 2011/0144421, U.S. 2011/0226262, and U.S. 2012/0132214, may be used.

Implant 100 also has a third anchor 112. In this embodiment, the third anchor holds the anterior end of the implant in place. The third anchor may be fixed in position, such as in an airway forming tissue or a mandible of a patient. Once anchored in place in a tissue, an anchor exerts a tension force on the tissue. The first, second, and third anchors may be placed in the same type of tissue (e.g. tongue, soft palate, pharyngeal tissue) or may be placed in different types of tissue. The first, second, and third anchor ends may be the same type of anchors, or may be different types of anchors. Any type of biocompatible (or covered or coated to be biocompatible) anchor ends that are able to hold the implant in a desired location in a tissue may be used, such as those described in U.S. Pat. No. 8,167,787 and U.S. Patent Publication No. 2011/0144421. FIG. 1B shows relative movement of an element and legs, such as in response to a force. A force pulls on anchor 108 and causes elongate member 110 to at least partially rotate around the wheel portion of pulley 114, extending second elongate leg 104′ in the direction shown by arrow 130 and shortening first elongate leg 102′ in the direction shown by arrow 132. Rotation of elongate member 110 around pulley 114 redistributes the force or tension and reduces peak force on the elongate member compared with the force that would be experienced by an elongate member that does not rotate.

Pulley 114 is connected with anchor 112 by axis 140. Anchor 112 holds the axis and pulley in place. Axis 114 may be any shape or dimension that anchors pulley 114 to anchor 112.

FIG. 1C shows an alternate implant embodiment for redistributing a peak force. An elongate member 120 pivots around eye-bolt 122 in response to a force, and extends towards the force, as shown by arrow 124. First leg 136 shortens in response.

An elongate member or axis (segment) may include a single type of material or may have portions or regions that include two (or more than two) types of materials. The elongate member and the axis can be made of any material or materials that are biocompatible (or coated or treated to be biocompatible) and provide sufficient strength. An elongate member or axis may include one material or may include more than one material. An elongate member or axis material may be rigid or may be deformable, elastomeric, flexible, and/or resilient. An elongate member may be, for example, one or more of a metal or a polymer; a suitable metal may include any one or more of spring steel, stainless steel, or superelastic nickel-titanium alloy and a suitable polymer may include any one or more of polyesters, polyolefins, polyurethanes, and/or silicon rubber. An elongate member may be one material, or may have more than one material (e.g. two, three, four, five, more than five, or more than ten different types of materials).

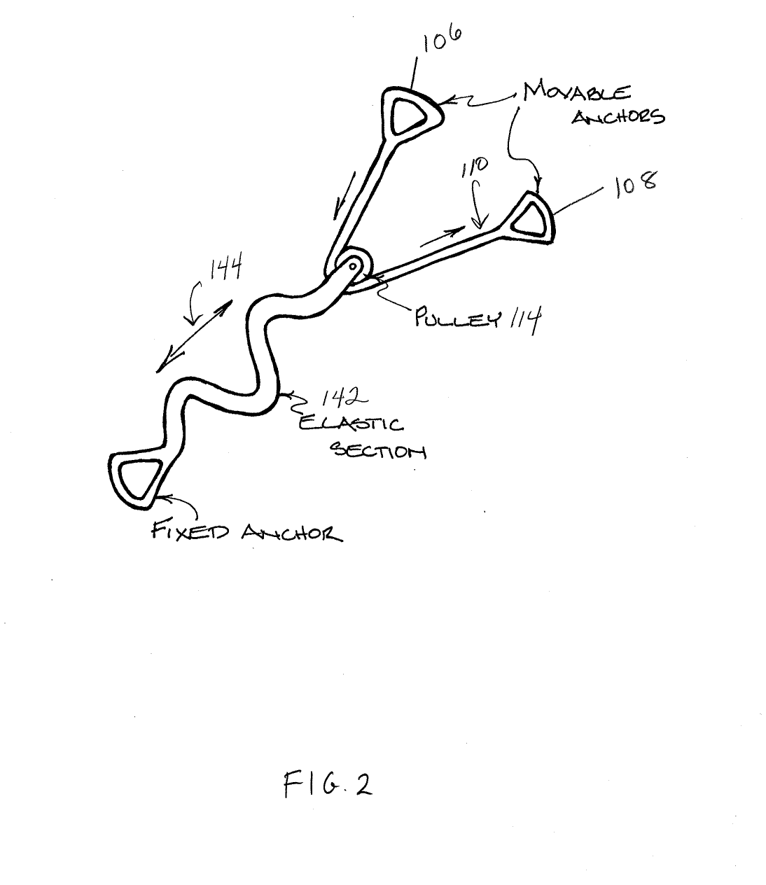

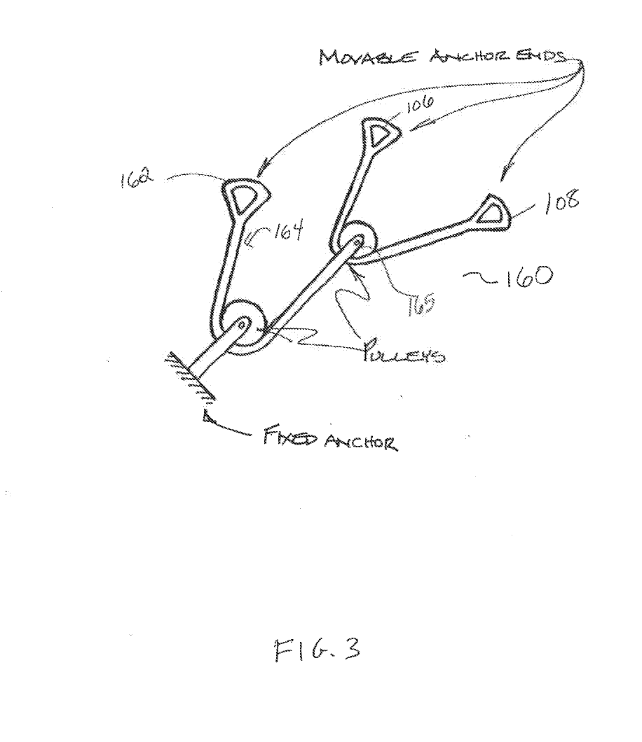

FIG. 2 shows another embodiment of an implant for redistributing a (peak) force. The implant has first end 106 and second end 108 that rotates around pulley 114 to redistribute a force, such as one exerted on anchor end 108. Pulley 114 is connected with elastic axis 142, which is shaped and configured to expand and compress in the direction shown by arrow 144. A peak force may be redistributed by elastic axis 142 in addition to, or instead of, being redistributed by movement/rotation of the elongate member 110 about pulley 114.

FIG. 3 shows another embodiment of an implant for redistributing a (peak) force or tension. Implant 160 includes a plurality of anchor ends for anchoring the implant to tissue and effecting tissue (e.g. airway forming tissue) movement. Curved axis 164 redistributes force between two sets of elongate members, the 1^{st} set having anchor end 106 and anchor end 108, and the second set having anchor end 162 and pulley 165. Having a plurality of elongate members, a plurality of anchor ends, and/or a plurality of pulleys in a single patient may be done for any reason. It may improve force redistribution and prevent tissue tearing or damage (as discussed above). It may allow more modulated force to be experienced by a tissue. It may allow tissue movements that better mimic natural (or desired) tissue movements.

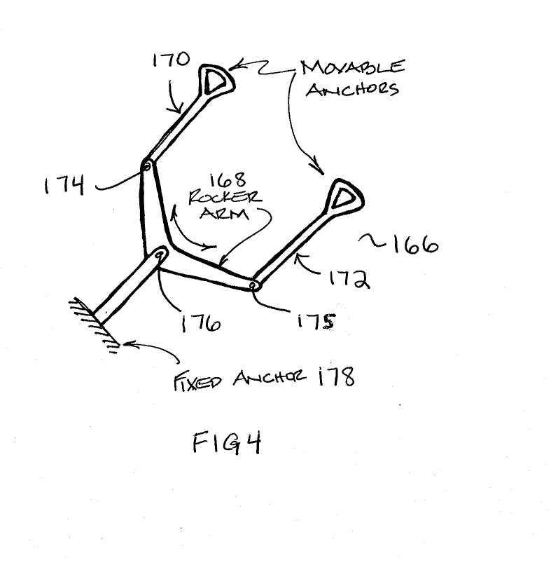

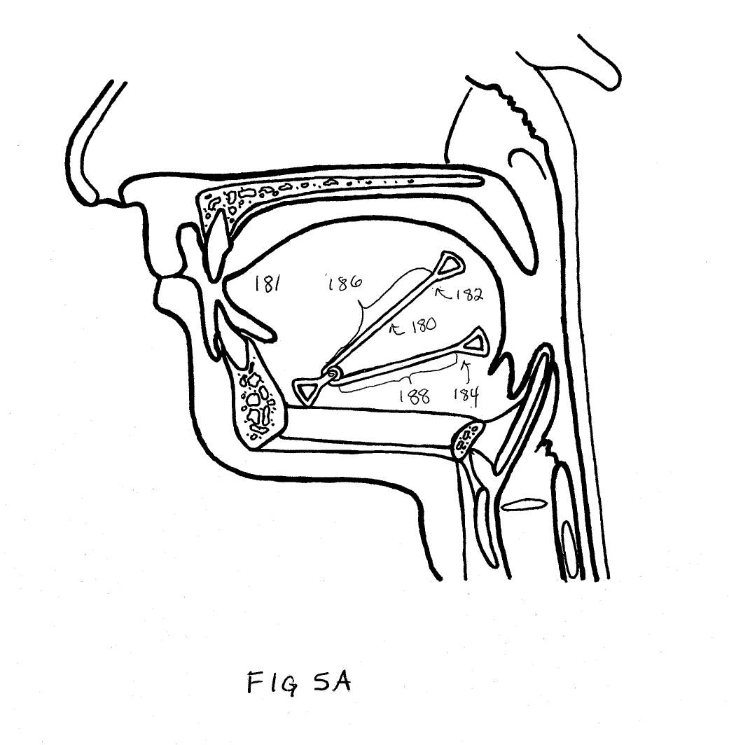

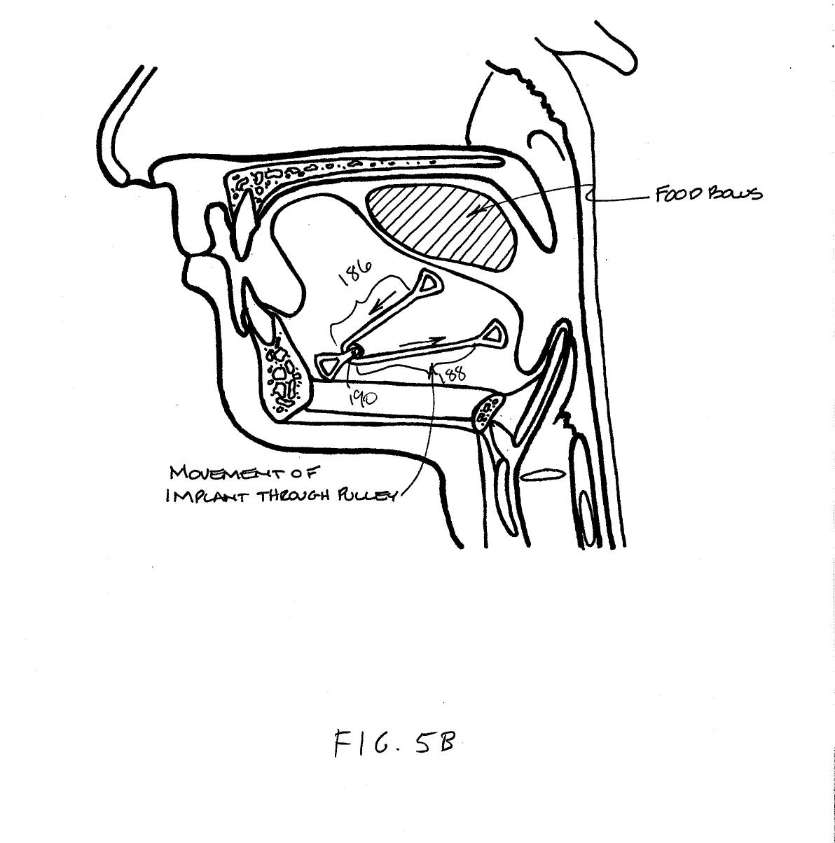

FIG. 4 shows another embodiment of an implant for redistributing a peak force or tension. First elongate leg 170 and second elongate leg 172 are connected with rocker leg 168 at first point 174 and second point 175. Rocker leg 168 in turn is connected with anchor 178 at third point 176. Any or all (or none) of the first, second, and third points may pivot in order to redistribute a force and/or anchor an implant into a preferred position.

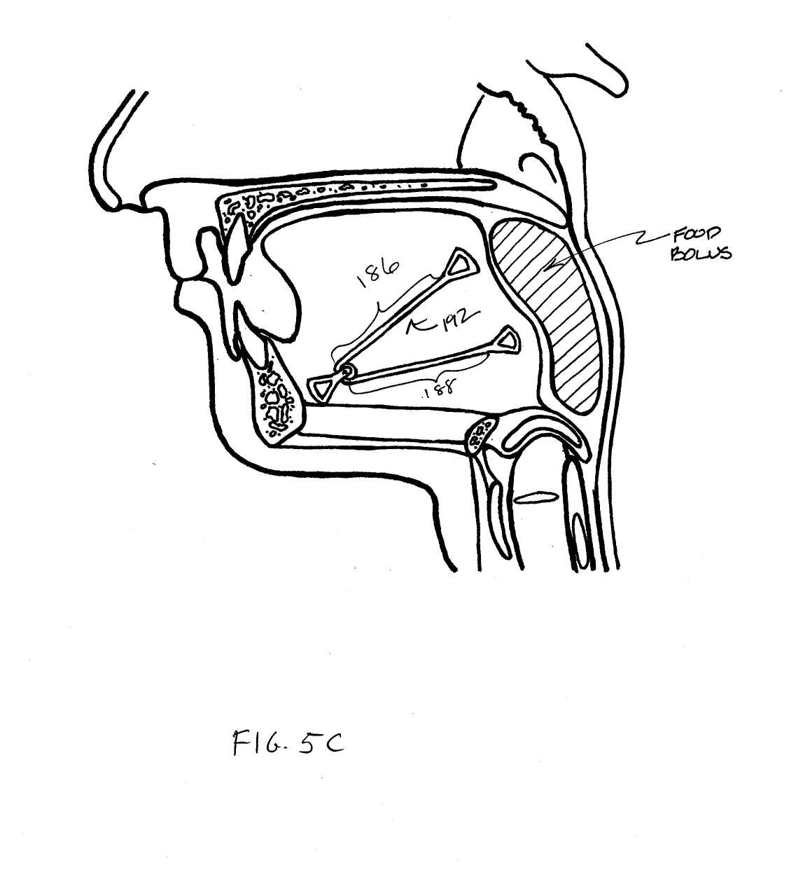

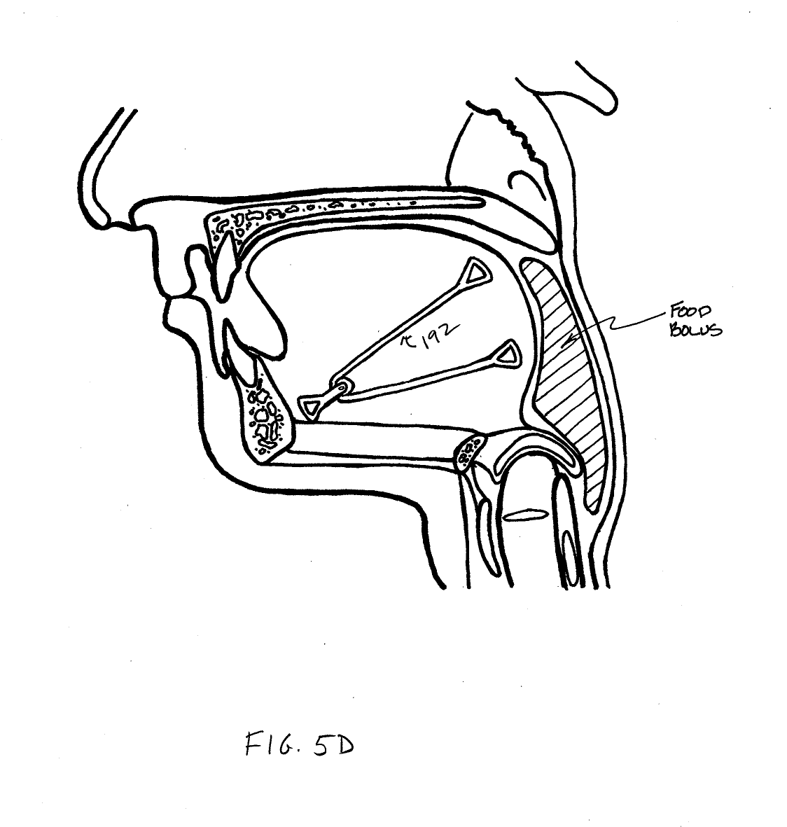

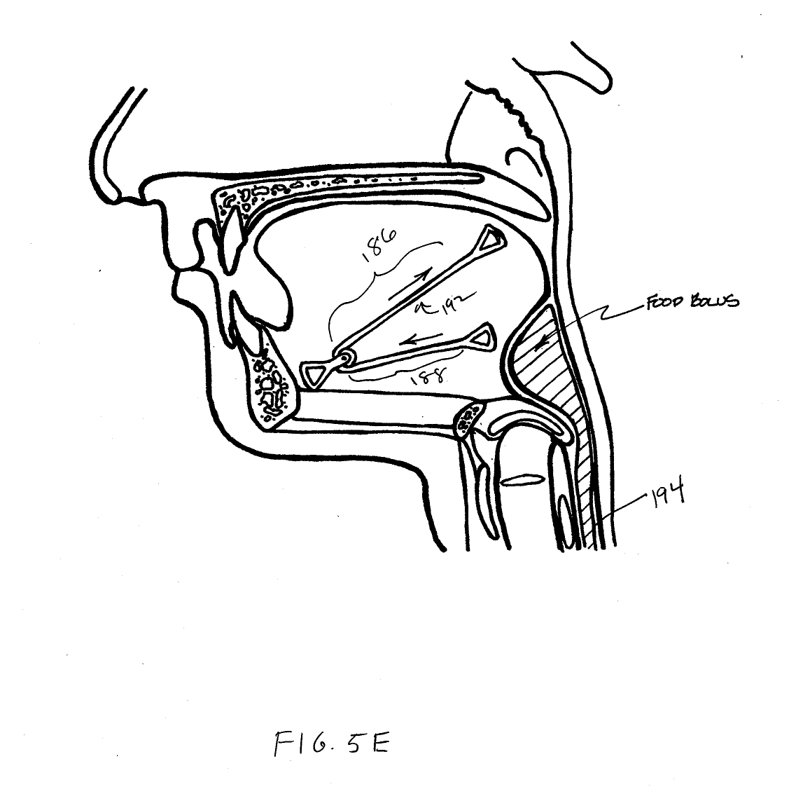

FIGS. 5 A-E show an accommodating implant placed in tongue 181 of a patient and how the implant may aid in swallowing or accommodate a bolus of food as a wave of tongue movement passes food through the mouth to the esophagus. FIG. 5A shows implant 180 in place in a tongue of a patient, such as when the tongue is at relative rest. First elongate leg 182 and second elongate leg 184 experience similar amounts of force and are about the same length. As food is moved to the back of mouth, the tongue reshapes to accommodate the bolus of food and to force the bolus along the back of the mouth. In FIG. 5B, pulley 190 redistributes force from the first elongate leg 186 to the second leg 188 to move the bolus of food. In FIG. 5C, as the bolus moves to the back of the mouth, elongate member 192 is distributed more or less equally between first elongate leg 186 and second elongate leg 188. Finally, elongate leg 188 of elongate member 192 becomes shorter relative to elongate leg 186 as the food moves past the tongue and into esophagus 194, as shown in FIG. 5E.

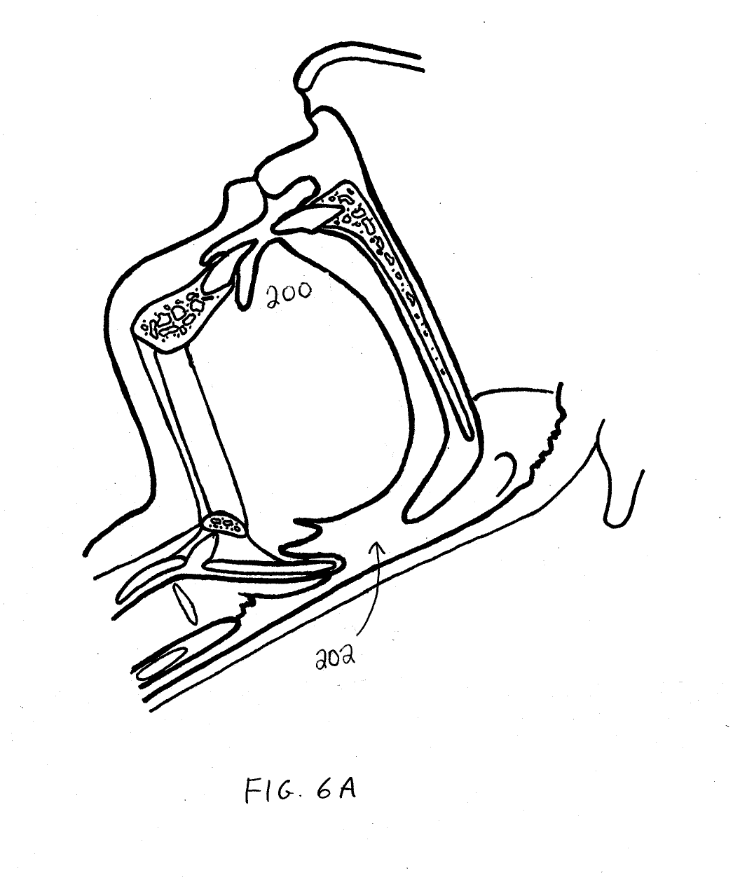

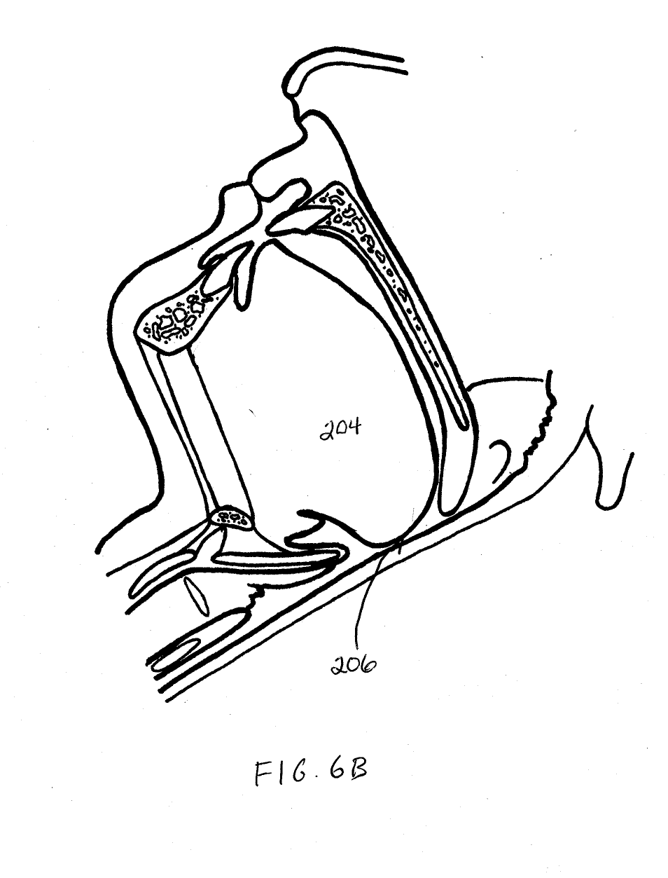

FIGS. 6A-C show how an implant according to the disclosure can be used to treat a patient for apnea or snoring. FIG. 6A shows tongue 200 of a normal person who is not in need of treatment. Airway 202 is open and air can pass freely from the mouth or nasal passages into the lungs. FIG. 6B shows a patient during a sleep cycle in need of treatment. Tongue 204 has fallen back and is obstructing airway 206. FIG. 6C shows the patient shown in FIG. 6B during a sleep cycle after implantation of an accommodating implant. Implant 208 exerts sufficient force on tongue 204′ to open up airway 206′ and allow air to pass and to prevent apnea and/or snoring.

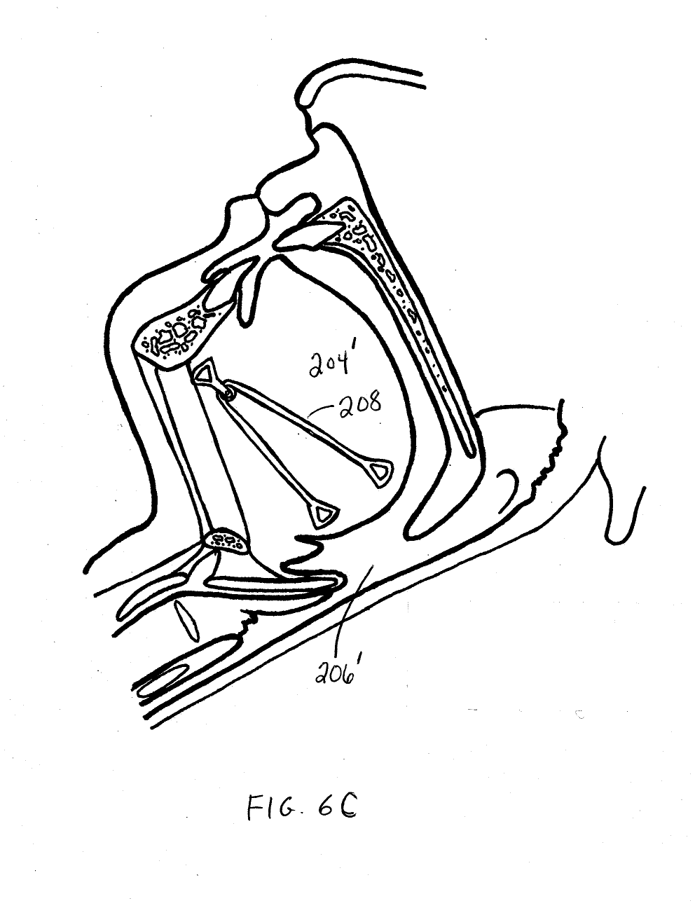

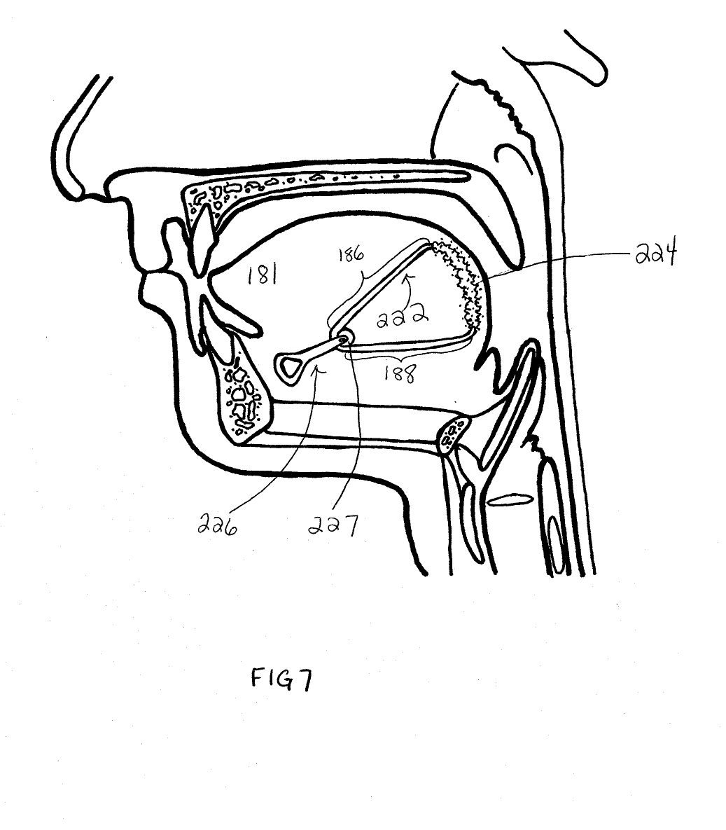

FIGS. 7-10 show other embodiments of accommodating implants that may be used for treating any sort of airway tissue disorder, problem, or syndrome. FIG. 7 shows implant 222 implanted in tongue 181, with anterior fixed anchor 226 and pulley 227 to redistribute a force between first elongate leg 186 and second elongate leg 188. Posterior anchor 224 connects with both first elongate leg 186 and second elongate leg 188. Posterior anchor 224 may be any length and any material. The anchor may be less than 0.5 cm, less than 1 cm, less than 2 cm, or less than 3 cm in a longest dimension (“length”). The anchor may be greater than 1 cm, greater than 2 cm, greater than 3 cm, or greater than 4 cm in a longest dimension (“length”). In one example, it may be a woven cloth or porous material configured to allow tissue growth or ingrowth. The anchor may anchor the implant while minimizing any cutting, shredding, or tearing of the tissue. The anchor may additionally aid in redistributing forces.

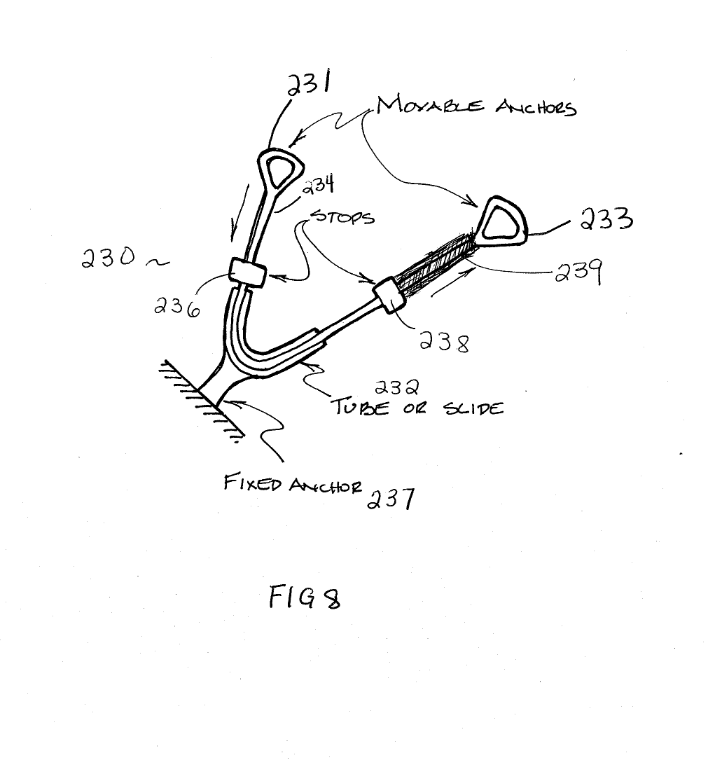

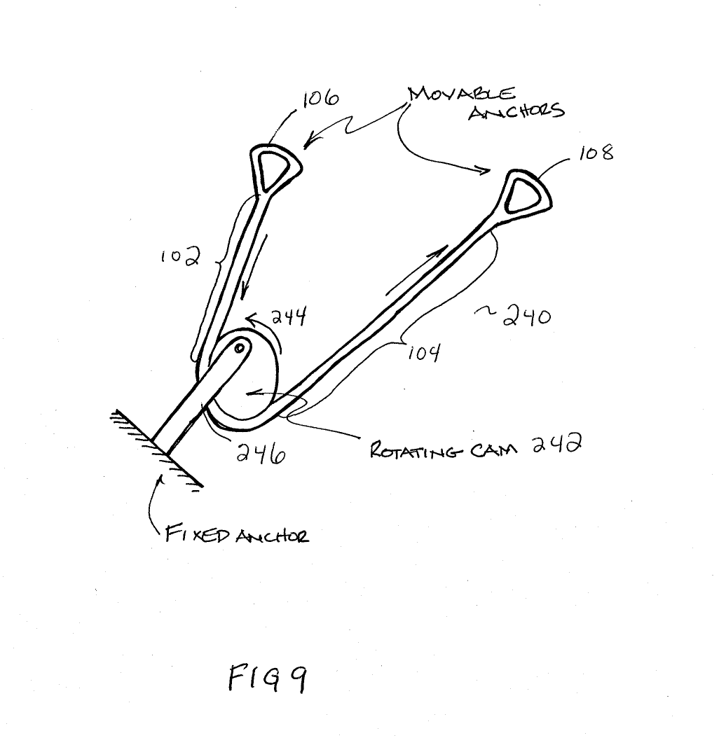

FIG. 8 shows another embodiment of an accommodating implant to redistribute a force. Implant 230 has slide member 232 that accommodates the elongate member 234. Slide member 234 may be any shape or have any openings or be made of any material that allow or aid elongate member 234 to move through it to accommodate a force placed on it. A slide member may prevent or reduce tissue catching, damage, or pinching. In one example slide member 234 may be made of a smooth, slippery material and/or may be coated to be smooth or slippery. It may be made of or coated with one or more of a ceramic, a metal, a polymer, or any other material or it may have a groove to accommodate an elongate leg. Elongate member 234 may have first stop 236 and second stop 238 to limit the amount of movement of the elongate member 234 through slide member 234. Implant 230 has a first, elongated configuration and a second, therapeutic configuration. The implant is held in the first, elongated position by bioerodible material 239. The bioerodible material provides a force that resists a compressive force to hold the implant in the first, elongated position by opposing at least part of second stop 238 and opposing anchor 233. Over time, anchors 231 and 233 anchor to tissue after placement of the implant in a tissue and a tissue plug grows through the loop of each anchor. During this time, very little or no force is transmitted from implant 230 to the surrounding tissue. After exposure to a body fluid or other treatment, the bioerodible material bioerodes, and the implant foreshortens. Fixed anchor 237 may be placed in its therapeutically useful position at the same time that the implant is place in a tissue, or it may be placed in its therapeutically useful position after time. In one example, the implant is implanted in a tissue, first anchor 231 and second anchor 233 are bioanchored due to tissue growth, and then fixed anchor 237 is manually pulled to its therapeutic position in a second procedure, wherein the fixed anchor 237 may be attached to tongue, palate, or bone (e.g. the mandible).

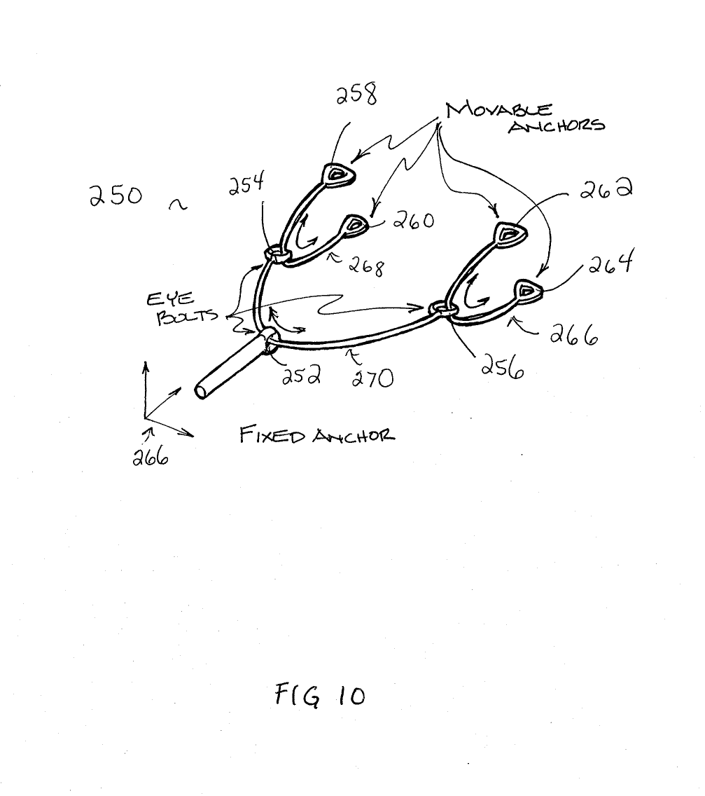

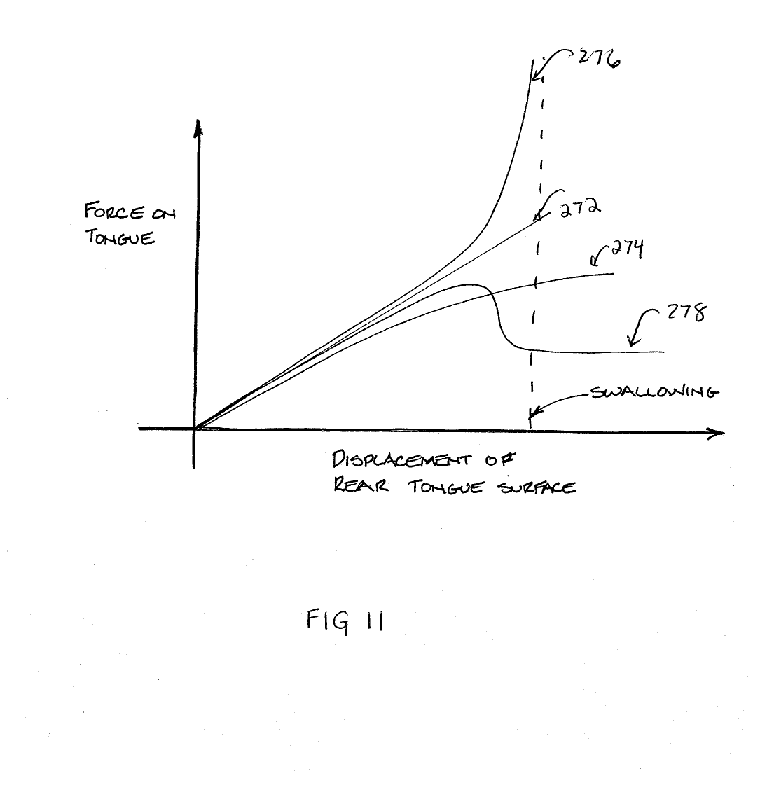

FIG. 9 shows another embodiment of an accommodating implant. Implant 240 includes cam 242 configured to create a mechanical advantage between first elongate leg 102 and second elongate leg 104 of elongate member 110. A modified slide member (not shown), similar to the one shown in FIG. 8, may be placed over the cam and/or part of elongate member 110. A modified slide member may aid elongate member 110 in moving along the cam and/or prevent or reduce tissue catching, damage, or pinching.

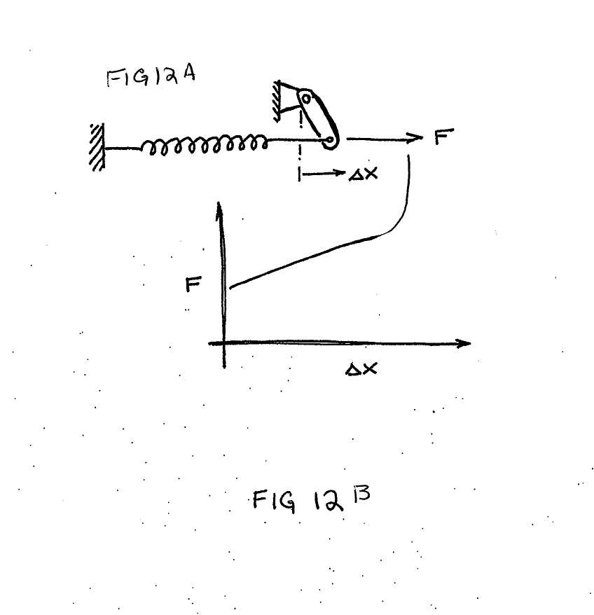

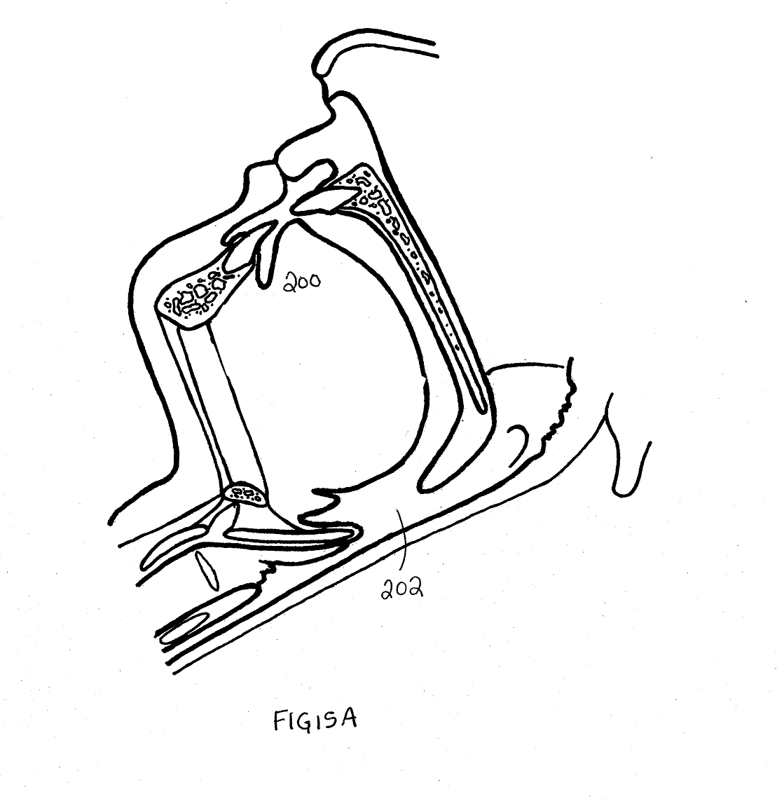

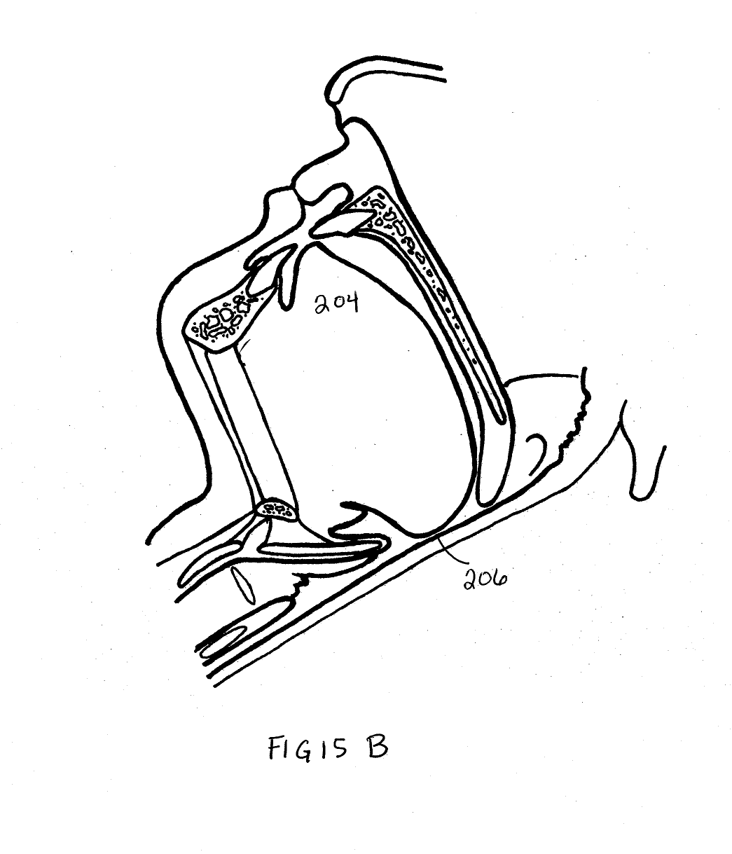

FIG. 10 shows a device with a plurality of anchor ends, elongate members, and pivot points that can be implanted to provide and to redistribute force in the X, Y, and/or Z directions as shown on coordinate system 266 using cascading, adjustable elements. Eyebolts 252, 254, and 256, elongate legs 266, 268, and 270, and anchor ends 258, 260, 262, and 264 can be placed in a single plane, or can be placed in different planes or at different levels. Two (or more or all) elements may be placed in the same or in different cranial-caudal levels relative to a patient's body (e.g. different distances from a transverse plane relative to a patient's body, different anterior-posterior positions, and/or different lateral distances from a midline plane of a patient or tissue (such as a tongue, palate). Instead of, or in addition to an eyebolt, any mechanism that allows elongate legs to move through or rotate about it can be used, such as pulleys or slides, or a combination of thereof. Additionally, or instead, a solid mechanism, such as shown in FIG. 14 may be used to distribute forces and may be placed to redistribute forces in the X, Y, and/or Z directions.

FIG. 11 shows several curves of force that can be applied to the tongue as a function of displacement of the rear tongue surface. Controlling the amount of force applied to an implant (and in turn the amount of force applied to a tissue being treated), allows implants to be chosen that will balance forces at different times. In particular, it is desirable during swallowing to redistribute forces or to otherwise reduce force on part of an implant (and part of a tongue) to allow or cause tongue movement to aid in swallowing. It is desirable during sleep to provide sufficient force on an implant to reduce or prevent the tissue (e.g. tongue) from falling back and otherwise creating an apneic event, a hypopneic event, a snoring event, or some other undesired event. Curve 272 shows an implant that provides a relatively constant rate of force, such as a spring (e.g. a spring or material that behaves like a spring). Curve 274 shows an implant that provides a lower constant rate of force, such as a softer spring. Curve 276 is a material that stiffens significantly upon swallowing after being placed in a tongue. Curve 278 shows the force from a material that provides a non-linear force, including a stiffening effect similar to that provided by a spring over a first range of tongue movements, but which drops off sharply to aid or allow swallowing during a second range of tongue movements.

FIG. 12A shows a model that can be used to control an amount of force as force is modulated between a spring and a lever arm. As the spring is stretched and the arm displaced (AX), the relative amount of force (F) experienced changes. FIG. 12B shows an amount of force in which the force increases as a function of displacement.

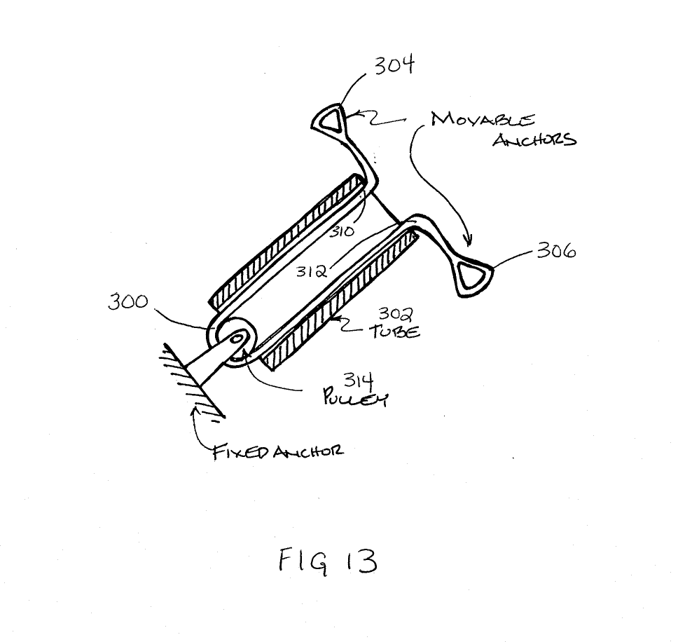

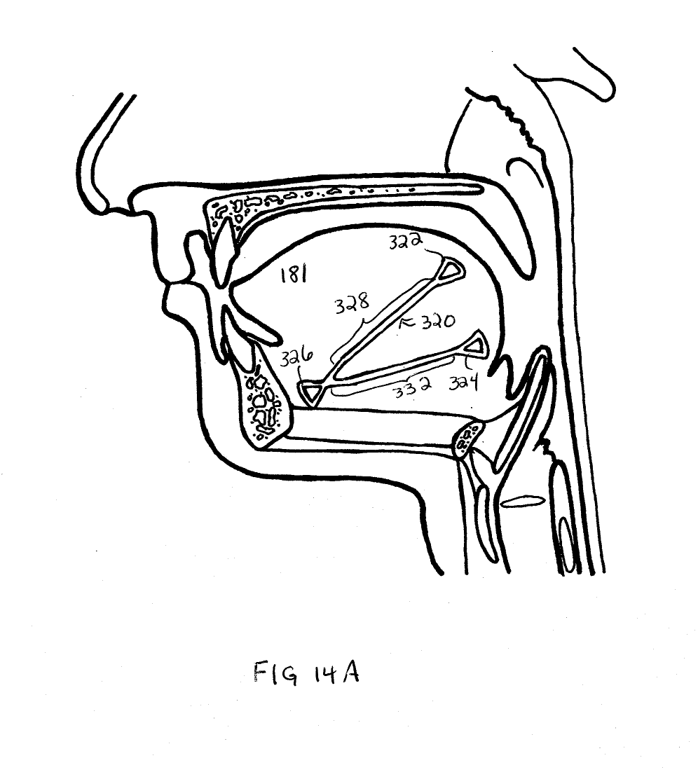

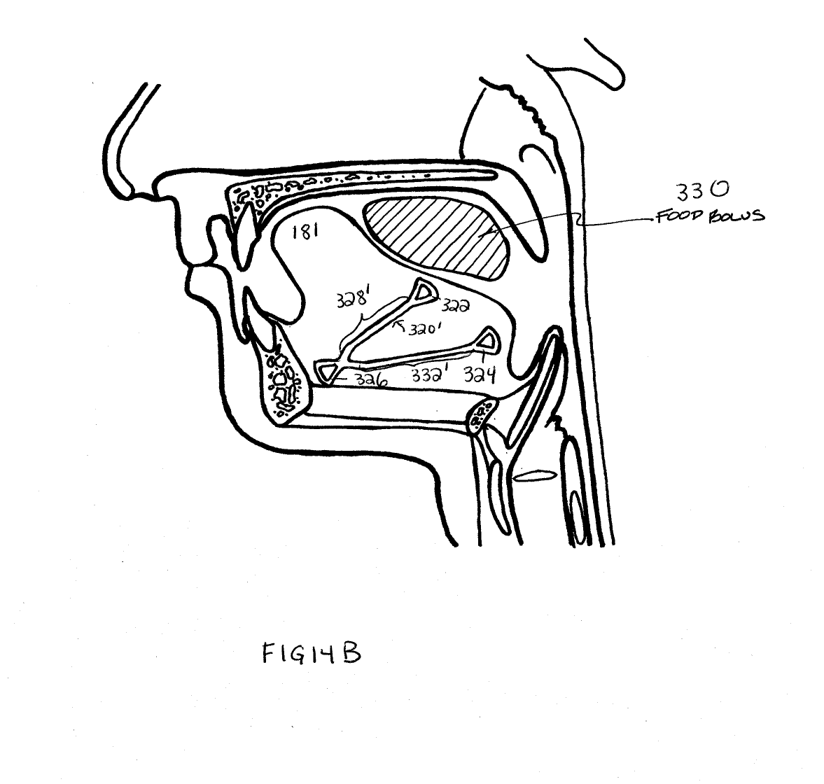

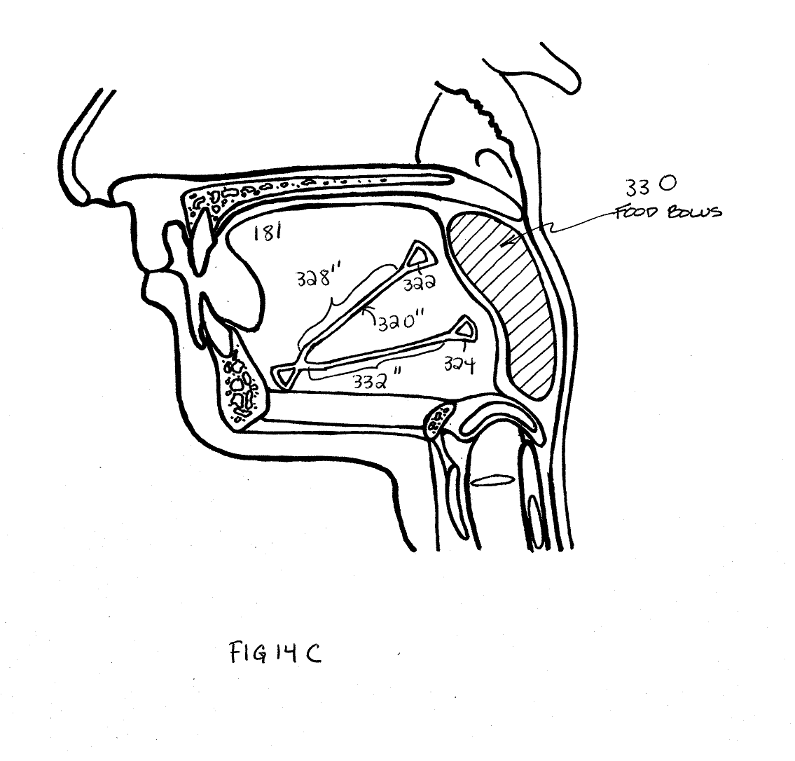

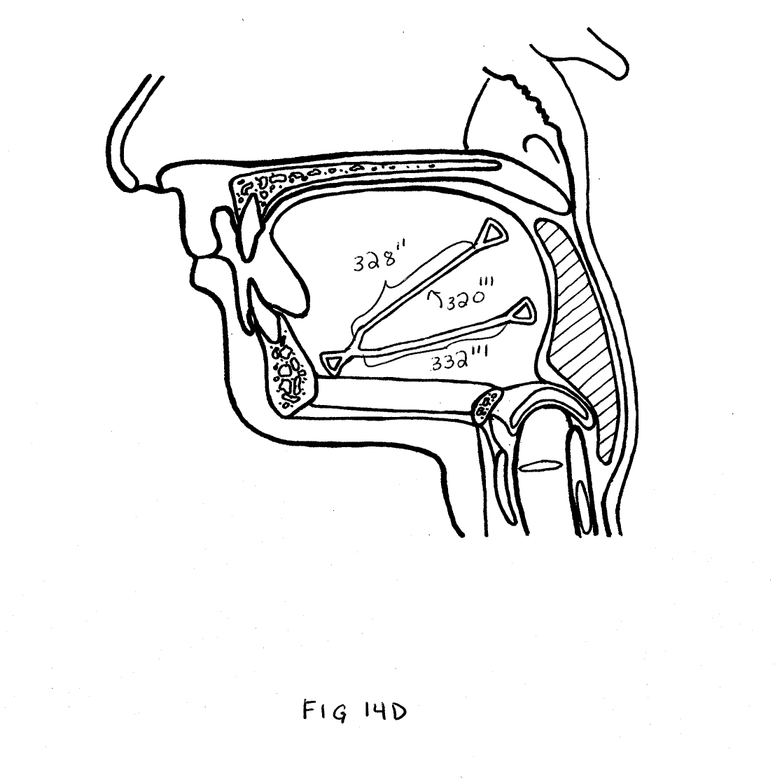

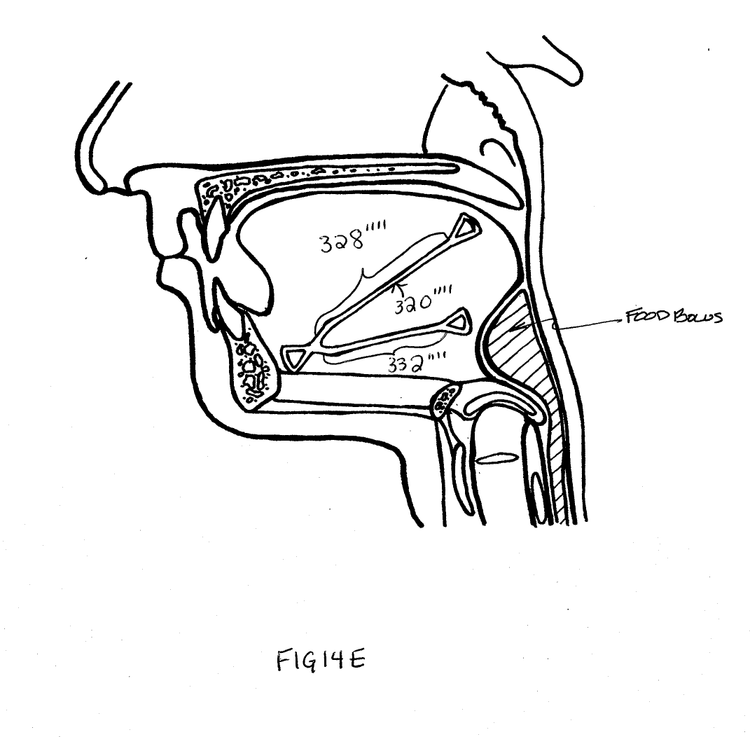

FIG. 13 shows an implant with a means to create a directionally controlled motion of adjustable left anchor 304 and adjustable right anchor 306. Elongate element 300 rotates around pulley 314 while tube 302 creates a right angle in left leg 310 and a right angle in right leg 312.

FIGS. 14 A-E show another example of how an accommodating implant may aid or better allow swallowing, similar to that shown in FIGS. 5A-E. Elastomeric implant 320 is anchored in a tongue of a patient at first anchor 322 (first posterior or movable anchor), second anchor 324 (second posterior or movable anchor), and third anchor 326 (anterior or fixed anchor). The elastomeric implant can be made from one or more of the materials described above. As the tongue moves bolus of food 320 through the mouth and along the tongue and into the esophagus, it undertakes a series of shape changes to aid in the movement. The shape changes can be modeled as a wave passing along the tongue. The accommodating implants change shape to accommodate the changes in the shape and forces in the tongue. Initially first leg 328 and second leg 332 of elastomeric implant 320 are about the same length and are subject to the about the same amount of force. As the tongue moves the food to the back of the mouth, as shown in FIG. 14B, first leg 328′ may be subject to a compressive force and/or second leg 332′ may be subject to an expansive force, and the first and second leg (mediated by the third anchor) redistribute the force(s), with first leg 328′ shortening and second leg 332′ lengthening. The redistribution of force may reduce forces on any part(s) of the implants, including any of the legs and anchor ends. The redistribution may also reduce force on the tongue tissue to which the anchors are attached, which may minimize side effects such as pain or tearing. As the tongue moves the food to the back of the mouth, the forces acting on the first leg and second leg start to equalize, and the lengths of the first leg 332″ and second leg 328″ equalizes as shown in FIG. 14C. Finally, as the tongue moves the food into the esophagus, the relative forces on the first and second legs of the tongue are reversed, and the second leg 332″″ foreshortens as the first leg 328 lengthens in response, as shown in FIGS. 14 D-E.

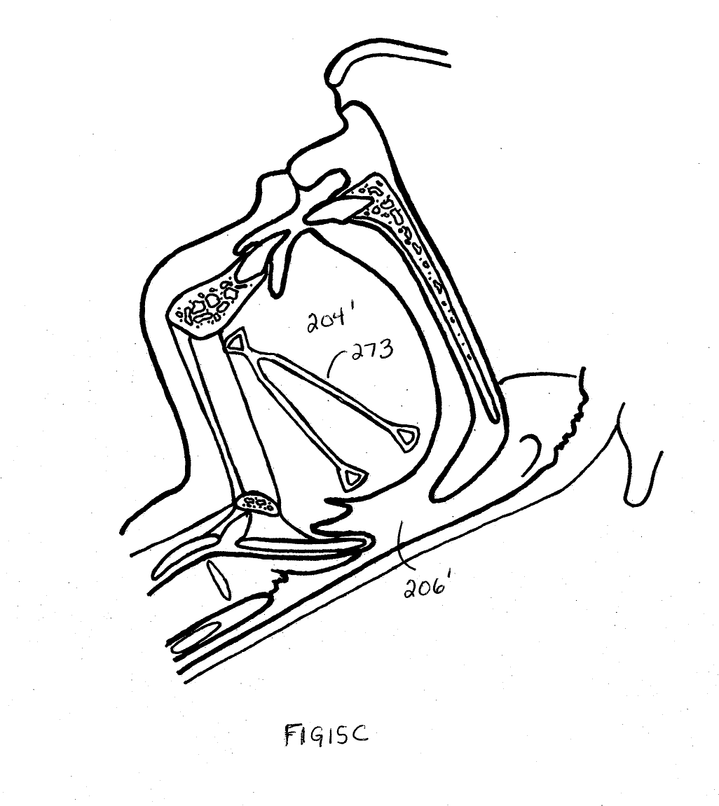

FIGS. 15A-C show how an implant according to the disclosure can be used to treat a patient for apnea or snoring similar. FIG. 15A shows tongue 200 of a normal person who is not in need of treatment. Airway 202 is open and air can pass freely from the mouth or nasal passages into the lungs. FIG. 15B shows a patient during a sleep cycle in need of treatment. Tongue 204 has fallen back and is obstructing airway 206. FIG. 15C shows the patient shown in FIG. 15B during a sleep cycle after implantation of an accommodating implant. Implant 273 exerts sufficient force on tongue 204′ to open up airway 206′ and allow air to pass and to prevent apnea and/or snoring.

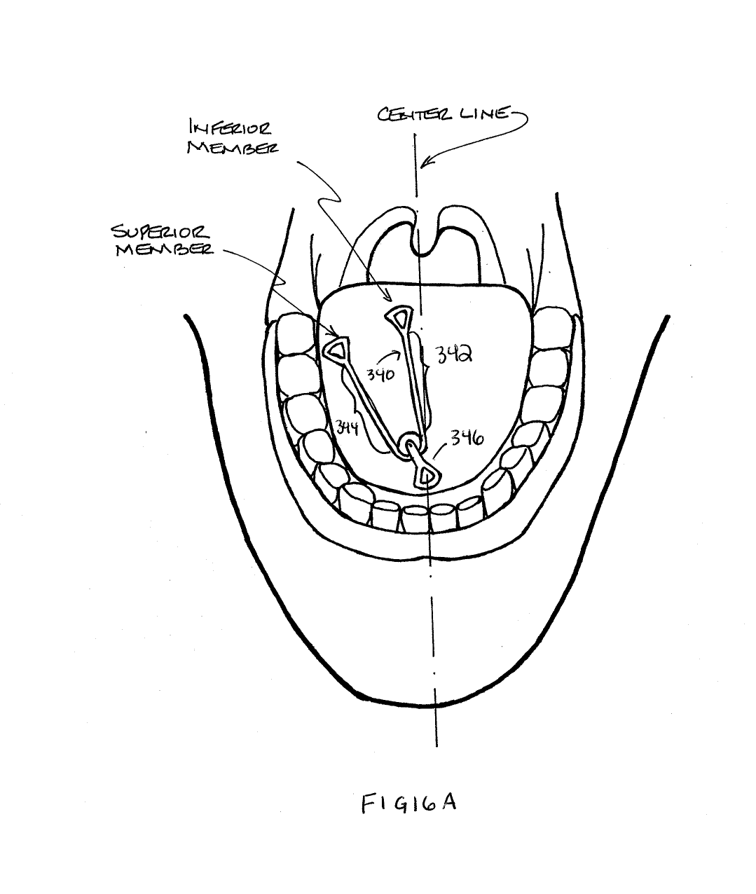

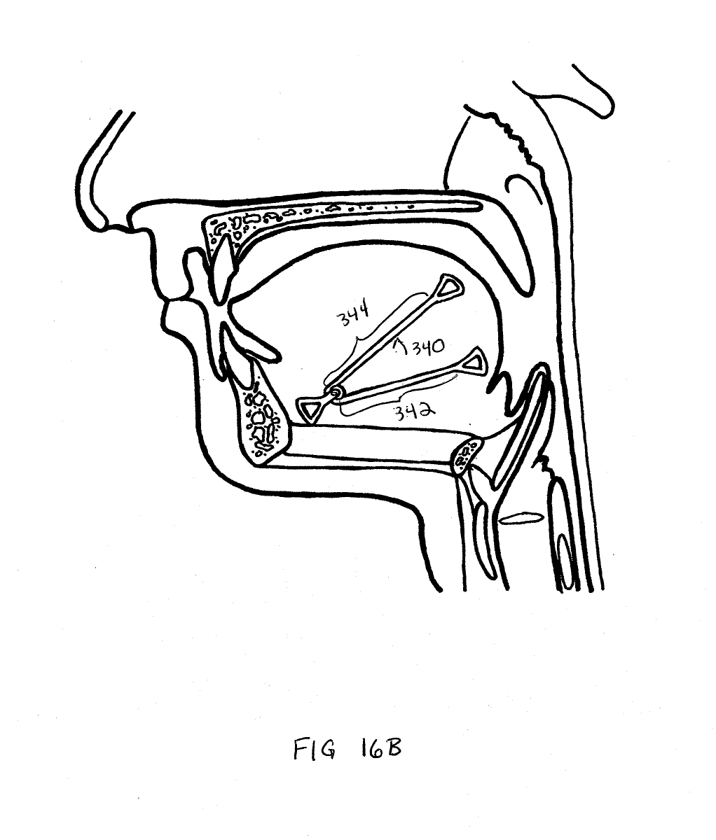

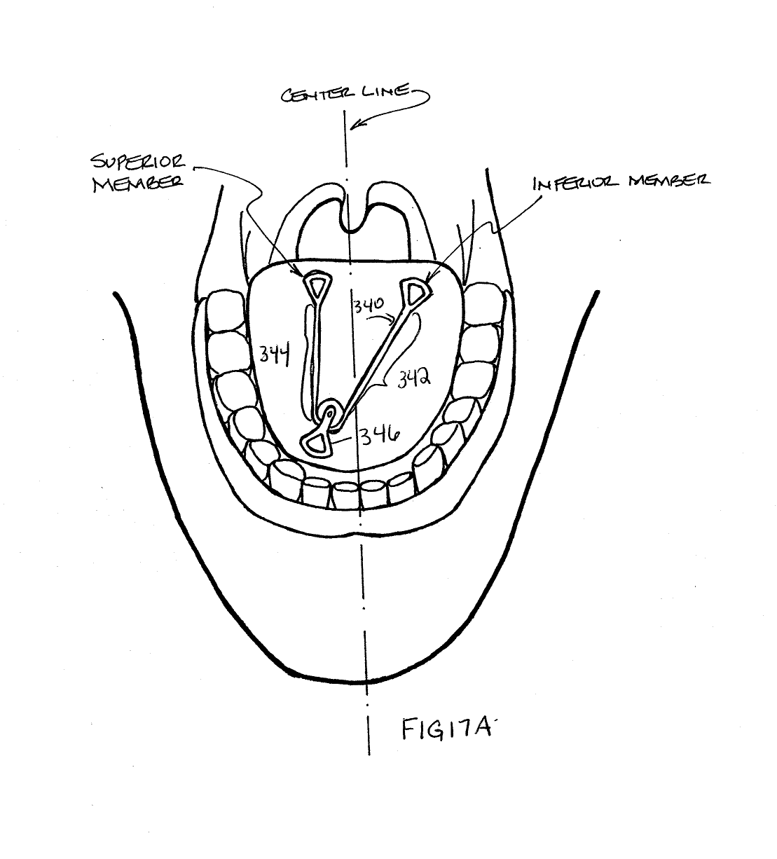

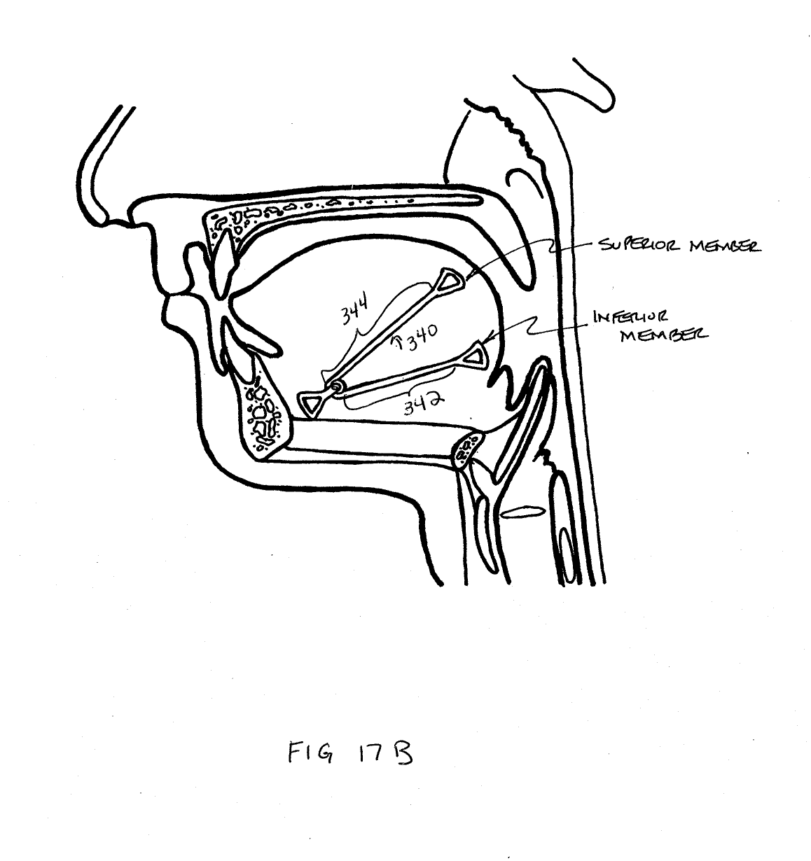

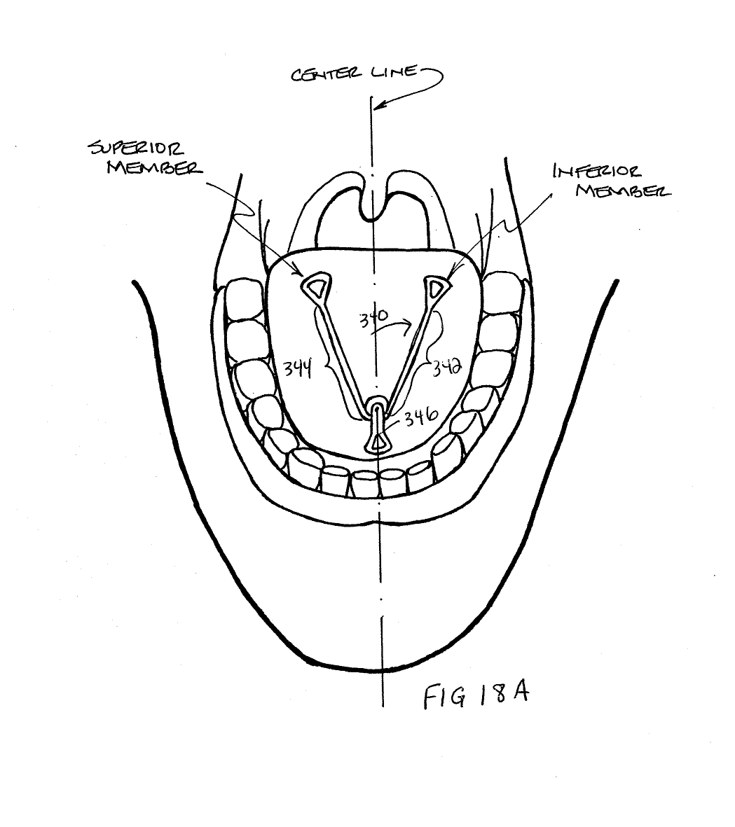

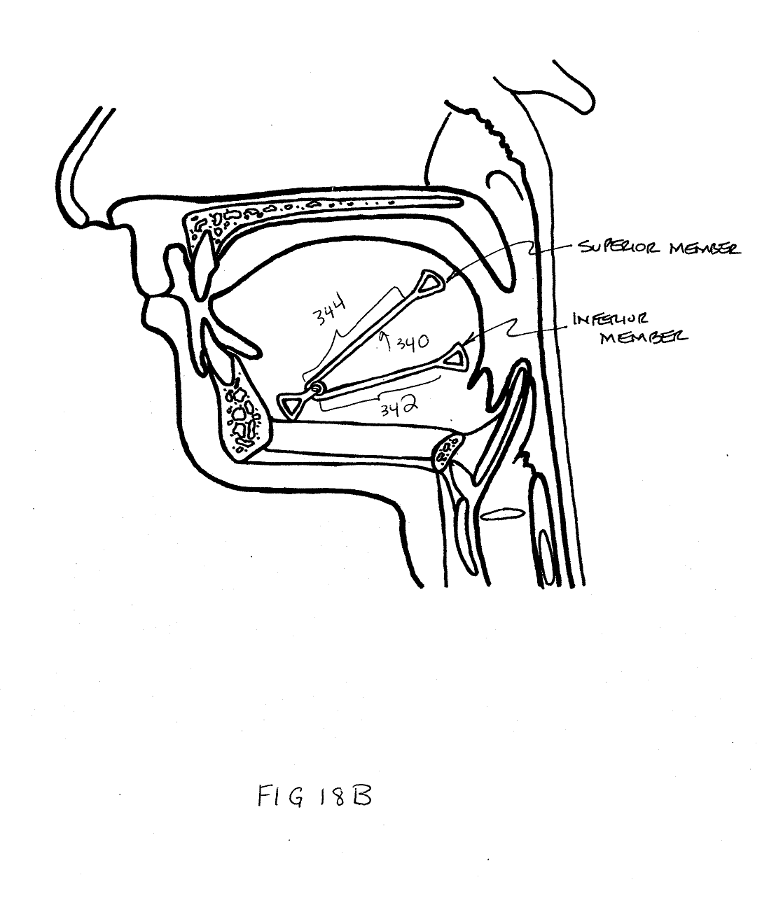

FIGS. 16A-B, 17A-B, and 18A-B show implants placed in various positions from a perspective view (FIGS. 16A, 17A, and 18A) and the same implants from a lateral or side view (FIGS. 16B, 17B, and 18B). First leg 344 (and its associated anchor) and second leg (and its associated anchor) of implant 340 are shown at different heights from a transverse plane of the body (e.g. different superior/inferior height positions) but could also be at the same height. First leg 344 and second leg 342 are shown on the same side (FIG. 16) and on opposite sides (FIGS. 17 and 18) of a midline plane of the body. A third anchor 346 may be on one side of the midline plane, on the other side of the midline plane, or directly on the midline plans. Although shown for implants with a pulley, any implant may be placed as described. An implant may be placed in any position or any orientation, and one, two, or more than two implants may be placed in a tissue.

### Example 1

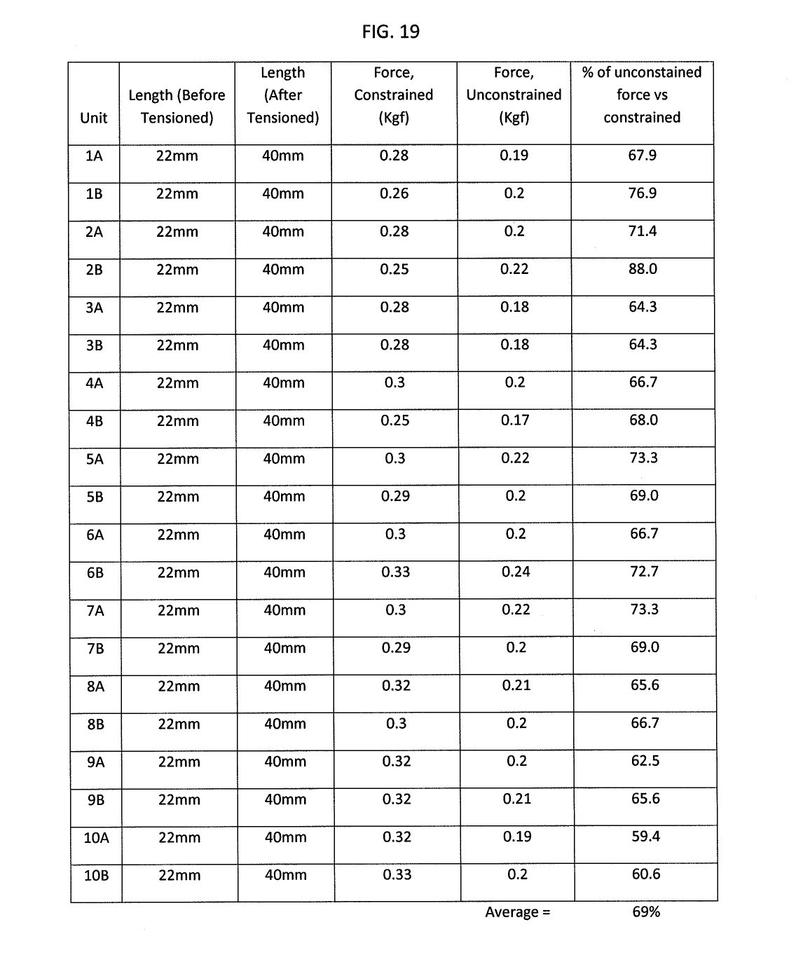

The force exerted by a v-shaped implant as it was rotated around a fixed point was compared to the force of an implant that was constrained and pulled in one direction. 10 V shaped implants were fabricated. Each implant was first tested in a constrained condition in which the apex was pinned down to prevent movement. Each leg of the implant was pulled from its original length of 22 mm to a length of 40 mm using a force gauge. The maximum force was recorded for each side of the implant. The apex was released and placed so that it could rotate around a fixed pin. One leg of the implant was pinned down to its natural position while the opposite leg was pulled using a force gauge from its original length of 22 mm to a length of 40 mm. Once again, the maximum force was recorded. The process was repeated on the opposite leg of the implant. The results of the restrained vs. unrestrained condition were compared and the average of all values calculated. The results are shown in FIG. 19. The unconstrained condition of a v shaped implant when given the chance to rotate around a fixed point will result in a force that is 69% of the force of the same implant leg in a constrained condition.

In variations of any of the foregoing embodiments, any, all or none of the legs or members spanning between the anchors may be formed from or include a rigid portion (such as a rod), a non-extendable portion (such as a wire), a resiliently stretchable portion (such as an elastic band), and/or a bioerodable portion (such as element 239 shown in FIG. 8).

As for additional details pertinent to the present invention, materials and manufacturing techniques may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

What is claimed is:

1. A self-adjusting implant comprising:
a first anchor configured to anchor to a first tissue location and apply a first tension force thereto;
a second anchor configured to anchor to a second tissue location spaced apart from the first tissue location and apply a second tension to the second tissue location;
a third anchor configured to anchor to a third tissue location spaced apart from and non-collinear with the first and the second tissue locations;
a first elastic tension-bearing segment spanning between the first and the third anchors, comprising a first deformable portion having a first elongated shape and a second contracted shape; and
a second elastic tension-bearing segment spanning between the second and the third anchors, wherein the third anchor is configured to generally distribute a tension of the first elastic tension-bearing segment with a tension of the second elastic tension-bearing segment.

2. The self-adjusting implant of claim 1 wherein the first deformable portion comprises an elastomeric material.

3. The self-adjusting implant of claim 2 wherein the elastomeric material comprises silicone.

4. The self-adjusting implant of claim 1 further comprising a material holding the first deformable portion in the first elongated shape.

5. The self-adjusting implant of claim 4 wherein the material comprises a bioerodible material holding the first deformable portion in the first elongated shape.

6. The self-adjusting implant of claim 4 wherein removal of the material allows the first deformable portion to transition toward the second contracted shape.

7. The self-adjusting implant of claim 1 wherein the second elastic segment further comprises a deformable portion configured to have a first, elongate shape and a second contracted shape.

8. The implant of claim 1 wherein the first and the second elastic tension-bearing segments are formed by a continuous elongate member.

9. The implant of claim 8 wherein the third anchor comprises a hole configured to slidably receive the continuous elongate member.

10. The implant of claim 2 wherein the third anchor comprises a pulley configured to movably receive the first deformable portion.

11. The implant of claim 1 wherein the third anchor comprises a slide member having an opening configured to allow the first elastic tension-bearing segment to move therethrough.

12. The implant of claim 1 wherein the third anchor is configured to generally equalize a tension of the first elastic tension-bearing segment with a tension of the second elastic tension-bearing segment.

13. The implant of claim 1 wherein the first and the second elastic tension-bearing segments are separate members that each terminate at the third anchor.

14. The implant of claim 13 wherein the third anchor comprises a lever.

15. The implant of claim 1 wherein the first and the second anchors are configured to be implanted in a patient's tongue near a base of the tongue, the third anchor is configured to be implanted in a region forward of the first and second anchors, and the first and the second elastic tension-bearing segments are sized to span between the base and the forward region.

16. The implant of claim 1 wherein at least one of the first and the second elastic tension-bearing segments is stretchable.

17. The implant of claim 1 wherein at least one of the first, second, and third anchors comprises a loop configured to permit tissue anchoring.

18. The implant of claim 17 wherein at least one of the first, second, and third anchors comprises a loop configured to permit tissue in-growth.

19. The implant of claim 1 wherein the third anchor comprises a stretchable segment having a first end and a second end, the first end being coupled to the first and the second elastic tension-bearing segments, and the second end being coupled to an anchor member configured to anchor to the third tissue location.

20. The implant of claim 1 wherein the third anchor forms a self-adjusting stage comprising a third tension-bearing segment, a fourth tension-bearing segment, a fourth tissue anchor and an anchor member configured to anchor the self-adjusting stage to the third tissue location, the third tension-bearing segment spanning between the first and the second elastic tension-bearing segments and the anchor member, the fourth tension-bearing segment spanning between the fourth tissue anchor and the anchor member.

21. The implant of claim 1 wherein the first, second, and third anchors are soft tissue anchors.

22. A method for treating an airway disorder, comprising:
implanting a first anchor of the self-adjusting implant at a first posterior tissue location;
implanting a second anchor of the self-adjusting implant at a second posterior tissue location spaced apart from the first posterior tissue location;
implanting a third anchor at a third tissue location spaced apart from and non-collinear with the first and the second posterior tissue locations,
wherein a first elastic tension-bearing segment spans between the first and third anchors and a second elastic tension-bearing segment spans between the second and third anchors, and the first anchor is configured to anchor to the first posterior tissue location and apply a first tension force thereto, the second anchor is configured to anchor to the second tissue location and configured to apply a second tension force thereto, and the third anchor is configured to generally distribute a tension of the first elastic tension-bearing segment with a tension of the second elastic tension-bearing segment, and the first elastic tension-bearing segment having an first elongated shape and a second contracted shape.

23. The method of claim 22 further comprising altering the length of the first elastic tension-bearing segment to redistribute the tension forces on the first and second elastic tension-bearing segments.

24. The method of claim 22 wherein the first posterior tissue location and the second posterior tissue locations are different vertical distances from a transverse plane of a patient.

25. The method of claim 24 wherein the different vertical distances differ by at least 1 cm.

26. The method of claim 22 further comprising redistributing the first or second tension force applied by either the first or the second anchors to prevent tissue damage by pivoting the first elastic tension-bearing segment or the second elastic tension-bearing segment.

27. The method of claim 22 wherein the implant comprises a plurality of pulleys.

28. The method of claim 22 wherein the first posterior location and the second posterior location are near a base of the tongue.

29. The method of claim 28 wherein the third tissue location is in a region forward of the first and second tissue locations.

30. The method of claim 22 further comprising distributing the tension of the first elastic tension-bearing segment and the tension of the second elastic tension-bearing segment by movably sliding at least one of the first elastic tension-bearing segment or the second elastic tension-bearing segment around a pulley coupled to the third anchor.

31. The method of claim 22, wherein the first elastic tension-bearing segment comprises a material holding the first elastic tension-bearing segment in the first elongated shape.

32. The method of claim 31 further comprising removing the material holding the first elastic tension-bearing segment in the first elongated shape to thereby allow the first elastic tension-bearing segment to transition toward the second contracted shape.

33. The method of claim 31, wherein the material is bioerodible.

34. The method of claim 33 further comprising permitting the bioerodible material to bioerode to thereby allow the first elastic tension-bearing segment to transition toward the second contracted shape.

35. The method of claim 22 wherein at least one of the first, second, and third anchors comprises a loop configured to permit tissue anchoring.

36. The method of claim 22 wherein the first elastic tension-bearing segment comprises an elastomeric material.
